(19) United States
(12) Patent Application Publication
(10) Pub. No.: US 2003/0125234 A1
(43) Pub. Date: Jul. 3, 2003

(54) Alteration of protein stability

(76) Inventor: Charles Russell Middaugh, Lawrence, KS (US)

Correspondence Address: HOVEY, WILLIAMS, TIMMONS & COLLINS, Suite 400, 2405 Grand, Kansas City, MO, 64108, US

(21) Appl. No.: 10/014,758

(22) Filed: Dec. 11, 2001

(51) Int. Cl. A61K 38/16; C07K 14/00; G01N 33/53

(52) U.S. Cl. 514/2; 435/7.1; 530/350

(57) ABSTRACT

A method of identifying compounds having effects on protein stability is provided. The method is capable of efficiently screening high numbers of compounds for their effects on a number of different proteins. Additionally, the present invention identified compounds and classes of compound which alter the stability of proteins. Compounds useful in the present invention include molecules having more than one charge. These molecules bind to unpaired charge sites on the surface of proteins, thereby altering the stability of the protein. The effect of the compounds on the protein is determined by the inhibition of protein aggregation attributable to the presence of the compound.

### BACKGROUND OF THE INVENTION

1. Field of the Invention

The present invention relates generally to proteins having altered stabilities, compounds which bind to proteins and alter the protein's stability, methods of altering the stability of proteins, and methods of identifying compounds which will alter the stability of proteins. More specifically, the present invention is concerned with compounds comprising molecules wherein the molecules have more than one charge and wherein the molecule binds to a protein, thereby altering the stability of the protein. Even more specifically, the present invention concerns di-ionic, tri-ionic, and tetra-ionic molecules which bind to unpaired charge sites on the surface of proteins, thereby altering the stability of the protein. Still more particularly, the present invention provides a method of screening large libraries of compounds to determine their effects on protein stability.

2. Discussion of Prior Art

Those ordinarily skilled in the art will appreciate that the alteration of the stability of proteins is a matter of significant practical importance. Decreases in protein stability are often manifested as aggregation and other physical changes in pharmaceutical formulations of proteins and in certain disease states such as Alzheimers disease, prion-mediated neurodegenerative disorders, and cataract formation. Protein stability is also a critical factor in the shelf life and subsequent utility of protein pharmaceuticals. Likewise, industrial enzymes are often significantly limited in use due to their lack of stability, especially at elevated temperature and such use could be expanded by altering their stability.

Two general approaches are commonly used to alter protein stability. The first involves the improvement of stability by the mutation of a protein's covalent structure through improved packing, introduction of disulfide bonds, addition of novel electrostatic interactions, or other chemical alterations. However, this approach is deficient in that it produces a new protein which may have undesirable properties, unrelated to the effects of the protein in its native state. In the case of pharmaceuticals, the new protein may possess undesired immunogenicity. The second approach involves the use of excipients to alter protein stability. While this method is often successful, it is limited by the current availability of a limited number of sugars, amino acids, polymers, and detergents that are deemed pharmaceutically acceptable based on safety considerations and previous experience with each such excipient.

Accordingly, what is needed in the art is a method of identifying compounds and classes of compounds which alter protein stability. A method of altering the stability of proteins which does not result in a new protein or produce undesirable immunogenic effects is also needed in the art. Additionally, what is needed are compounds which will bind to unpaired charge sites on the surface of proteins, thereby altering the stability of the protein. Preferably, such compounds will not interfere with the biological activity of the protein and will possess an acceptable safety profile.

### SUMMARY OF THE INVENTION

The present invention solves the problems of the prior art and provides a distinct advance in the state of the art by providing a new method of altering the stability of proteins and a new method of screening large numbers of compounds for their effects on protein stability. Additionally, the present invention provides a novel library of compounds which bind to proteins, thereby altering their stability.

The present invention is based on the fact that electrostatic interactions between side-chains on the surface of proteins often contribute significantly to protein stability. Moreover, inspection of the surfaces of most soluble proteins shows that they are often studded with many unpaired charged side-chains. Introduction of new charge pairs as well as the disruption of such interactions on protein surfaces are consistent with the important role of electrostatic interactions in protein stability. The physical instability of many proteins is thought to often be due to the appearance of molten-globule like states which are subject to self-association. Such states can be generated by a variety of environmental alterations and potentially provide “accelerated” stability information which can often be extrapolated to more modest conditions. These environmental alterations which may lead to destabilization and subsequent protein aggregation include elevated temperatures, low or high pH, and the presence of reducing, oxidizing, or chaotropic agents. In fact, destabilization can be induced by the alteration of any protein physical parameter that is sensitive to the conformational stability of the protein. Such alterations are commonly referred to as “stressing” the protein. These alterations can be detected by changes in a protein's intrinsic fluorescence emission spectrum, the binding of fluorescence dyes, circular dichroism, and many other methods which are well known in the art.

Thus, the present invention alters the stability of proteins by binding simple molecules containing multiple charges to the charged surface of a protein. It is presumed that the formation of a strong or weak complex between the compound and the native state of the protein increases the intrinsic stability of the protein. If the binding is not too strong as found here (k_{d}<10⁻⁶), the complex should dissociate into a bioactive protein and inert compound upon dilution into a biological milleau.

To select specific modifiers of protein stability, compounds are screened by observing the effect of these compounds under destabilizing conditions and comparing the stability of the protein without the compound to the stability of the protein with the compound. Preferably, the methods should be performable in a high throughput manner such as in a microtiter plate format. Once specific compounds are identified as being inhibitors or inducers of protein destabilization, their interactions with target proteins can be optimized using combinatorial chemistry based methods focused on either alterations of the terminal charged-groups or the intervening spacer regions.

Compounds particularly effective with the present invention comprise molecules which have more than one charge. Such molecules preferably include charges on either end (+/−, +/+, or −/−) that are spaced by flexible linkers of variable size. Compounds that employ amino and carboxyl terminal groups are spaced by either methylene or glycine residues. Preferably, the molecule is selected from the group consisting of di-, tri-, and tetra-ions. Di-, tri-, and tetra-ions that have been tested using methods of the present invention include oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, hexaglycine, ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃—NH₂-(Glu)⁻-(Gly)ₙ-(Glu)⁻-COO⁻, and combinations of these molecules wherein n can range from 1 to 5. Preferably, compounds used with the methods of the present invention have a molecular weight less than about 2000.

In some cases, the addition of the compound to the protein leads to an increase in protein stability while in other cases, the opposite effect occurs. Generally, the stability of the protein can be measured in a number of ways including determining the aggregation of the protein in solution, both with and without the compound. In order to speed up the destabilization process, a destabilizing agent or condition is added to the test protein system. One particularly convenient method of measuring aggregation involves determining the change of turbidity of the protein solution.

In one aspect of the present invention, a method of altering the stability of a protein is provided. This method generally involves contacting the protein with a compound which has more than one charge.

In another aspect of the present invention, a method of altering protein aggregation is provided. The method generally involves the steps of providing a protein in solution and contacting the protein with a compound which has more than one charge.

In another aspect of the present invention, a method of screening compounds for their effect on protein stability is provided. This method includes the steps of providing a protein in solution, adding a compound having more than one charge to the solution, adding a stability altering agent to the solution and determining the effect of the compound on the protein by measuring the alteration of protein stability. For this aspect, the determination of the compound's effect may include the step of testing the stability of the protein without the compound and comparing it to the stability of the protein with the compound. The addition of the stability altering agent may take place before, during or after the addition of the compound to the protein in solution.

In yet another aspect of the present invention, a protein having altered stability is provided. Such a protein comprises the protein having a ligand bound thereto. Preferably, the ligand comprises a molecule having more than one charge. Such a protein will exhibit a different stability when subjected to destabilizing conditions than is exhibited by the protein in its native state, that is without the ligand bound thereto.

In another aspect of the present invention, a ligand is provided. The ligand of the present invention has the general formula q-(X)ₙ-q wherein q represents a charged group or amino acid, X is another amino acid, methylene group or polyethylene glycol monomer unit, and n is from 1 to 6. Preferred amino acids include lysine, arginine, histidine, aspartic acid, and glutamic acid. Preferably such a ligand binds to a protein which is in its native state and which has unpaired charge sites on the surface thereof. More preferably, such a ligand will have a molecular weight of less than about 2000 except for the charged polyethylene glycol which may be much larger(n>>6). Optionally, the ligand may be modified at its N-terminus by methylation or some other neutral group or at its C-terminus by amidation or other neutral group.

Other aspects and advantages of the present invention will be apparent from the following detailed description of preferred embodiments and accompanying drawing figures.

### BRIEF DESCRIPTION OF THE DRAWING FIGURES

Preferred embodiments of the invention are described in detail below with reference to the attached drawing figures, wherein:

FIG. 1 is a graph illustrating the effect of a 10,000X molar excess of dianions on the aggregation of 1 mg/mL (7.1 μM) yeast alcohol dehydrogenase at 37° C.; and

FIG. 2 is a graph illustrating the effect of dianion concentration on the inhibition of FGF-1 aggregation.

### DETAILED DESCRIPTION OF THE PREFERRED EMBODIMENTS

The following example sets forth preferred methods and embodiments of the present invention. It is to be understood that this example is provided by way of illustration only and nothing therein should be taken as a limitation upon the overall scope of the invention.

### EXAMPLE 1

This example prepares the protein solutions and compounds used in the present invention as well as describing the tests used to determine the effects of the various compounds on each protein.

Materials and Methods

Preparation of Stock Protein Solutions.

Bovine insulin [Sigma, St. Louis, Mo., USA] was dissolved in phosphate saline (PBS) (50 mM sodium biphosphate, 100 mM sodium chloride), pH 7.0, bovine alpha-lactalbumin [Sigma] in 50 mM sodium phosphate, 100 mM sodium chloride and 2 mM EDTA, pH 6.2, chicken egg white lysozyme [Sigma] in 100 mM Tris, 1 mM EDTA, pH 8.2, human acidic fibroblast growth factor (FGF-1) in 6.2 mM sodium biphosphate, 120 mM sodium chloride, pH 7.2, porcine somatotropin (pST) [provided by Pfizer, Inc., Groton, Conn., USA] in 10 mM phosphate, pH 6. 1, human apolactoferrin (HLF) [Sigma] in 20 mM HEPES, 154 mM NaCl, pH 7.4, porcine heart citrate synthase (CS) in 40 mM HEPES, 50 mM KCl, 10 mM (NH₄)₂SO₄ mM potassium acetate, pH 7.8, and yeast alcohol dehydrogenase (ADH) [Sigma] in pH 7.0 PBS. Selection of buffer solutions was based on an extensive series of preliminary studies to obtain appropriate insolubilization kinetics as described below.

Preparation of Oxidizing/Reducing/Aggregation Inducing Agents.

Copper O-phenanthroline (1,10-phenanthroline) was prepared by mixing copper sulfate hexahydrate [Fisher] and o-phenanthroline [Sigma] in a 1:2 ratio. 1 M Dithiothreitol (DTT) was prepared at pH 7.0 PBS. Polyethylene glycol (M.W. 8000, Union Carbide, Danbury, Conn., USA) (PEG) was prepared at 20 mg/mL in 10 mM phosphate, pH 6.1.

Preparation of Bipolar Compounds.

The following compounds were obtained from Sigma and used without further purification: oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, and hexaglycine. Stock bipolar compound solutions were prepared by dissolving each in pH 7.0 PBS with the exception of triglycine, tetraglycine, pentaglycine, and hexaglycine due to their lack of solubility at pH 7.0, with the pH adjusted with hydrochloric acid/sodium hydroxide to 7.0. Triglycine and tetraglycine were dissolved in pH 8.6 PBS, while pentaglycine and hexaglycine were dissolved in pH 10.6 PBS.

The following charged peptides were synthesized by solid phase methods and their structure confirmed by mass spectrometry: ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃—NH₂-(Glu)⁻-(Gly)ₙ-(Glu)⁻-COO⁻ wheren=1 to 5. Each was either dissolved in pH 7.0 PBS, or pH 6.1 10 mM phosphate, and adjusted to the same pH. However, there was no discernable difference between the results obtained by peptides dissolved in PBS and those dissolved in 10 mM phosphate due to their adjustment to the same pH.

Procedure.

Protein aggregation was monitored for 1 hour at 360 nm using an HP8453 UV-visible spectrophotometer system [Hewlett Packard, Germany]. Preliminary studies established concentration ranges over which charged compounds exhibit inhibitory effects and solution conditions under which each protein displays appropriate aggregation behavior. Insulin (0.25 mg/mL (4.39E-5 M)) aggregation was monitored at 25° C. after addition of potential inhibitors and 20 μL of 1 M DTT (1.27E-4 M). Alpha-lactabumin (0.4 mg/mL (2.82E-5 M)) aggregation was monitored at 37° C. in the presence of 20 μL of 1 M DTT (1.27E-4 M). Lysozyme (0.4 mg/mL (2.86E-5 M)) aggregation was monitored at 25° C. in the presence of 20 μL of 1 M DTT (1.27E-4 M). FGF-1 (50 μg/mL (3.12E-6 M)) aggregation was monitored at 50° C. Porcine somatotropin (1.27 mg/mL (5.76E-5 M)) aggregation was monitored at 60° C. in the presence of PEG 8000 (20 mg/mL) which was included to enhance the protein association process through a simple exclusion volume effect. HLF (0.4 mg/mL (5.23E-6 M)) aggregation was monitored at 43° C. in the presence of 1.27E-4 M DTT. Citrate synthase (96 μg/mL (9.80E-7 M)) aggregation was monitored at45° C. ADH (1 mg/mL (7.14E-6 M)) was monitored at 37° C. following the addition of 7.76E-5 M Cu(OP)₂. Aggregation was compared to protein in the absence of the particular dipolar compound. After stirring, experiments were initiated by the addition of proteins followed by stressing reagents to 1 cm pathlength cuvettes employing a seven-position thermostatted sample holder that was cycled at 30-second intervals. Alternatively, a microtiter plate reader was employed under the same conditions in a 96 well format.

Data Analysis.

All optical density (OD)₃₆₀ₙₘ versus time results were exported into Excel [Microsoft] for data analysis. The maximum OD observed within 1 hour (O.D.ₘₐₓ) was used as the maximum extent of aggregation since aggregation was complete in each case by this time interval. The maximum rate of change in OD versus time plots (r) was used to determine the rate of the reaction from the slope at this point. An extrapolated line to the x-axis from the point of maximum change in OD was used to determine the delay time (τ) of the aggregation process. Inhibition of the charged species was then characterized by their ability to jointly affect maximum OD, delay time, and the rate of aggregation formation compared to a standard reaction without bipolar species present. On this basis, an inhibitory index (I) was defined as follows:

$$I = \frac{τ + / τ -}{( O . D {.}_{} + ) ( r + ) / ( O . D . - ) ( r - )}$$

where τ₊ and τ₋ are the delay times in the presence and absence of inhibitor, O.D.₊ and O.D.₋ are the maximum optical densities seen at 1 h in the presence and absence of inhibitor and r₊ and r₋ are the rates of time dependent turbidity change in the presence and absence of inhibitor, respectively.

Thus an inhibition index greater than 1 indicates that the charged species had an inhibitory effect on aggregate formation. A value of 1 implies no effect, while less than 1 indicates an enhancing effect on the aggregation process. Measurements were performed 3-5 times and standard derivations are reported.

Results.

Initially, conditions were established over which 8 representative proteins could be induced to aggregate over a one hour time period. The resultant turbidity (measured by the O.D. at 360 nm) versus time curves were characterized by distinct delay times before aggregation could be detected as well as by rates and extent of aggregation. To detect any effects of potential inhibitors of the various parameters of the aggregation process, an aggregation index that was proportional to the delay time but inversely related to the rate and extent of aggregation was defined. Thus, values greater than one are evidence of inhibitor activity while values of less than one are evidence of enhanced aggregation. Two types of libraries were selected for screening by this assay. The libraries are listed below in Table 1.

TABLE 1

| Name | Spacers |
|---|---|
| +NH₃—(CH₂)ₙ—COO⁻ | n = 2 − 7 |
| +NH₃—(CH₂)ₙ—NH₃⁺ | n = 0, 2 − 7 |
| ⁻OOC—(CH₂)ₙ—COO³⁻ | n = 0 − 7 |
| (Gly)ₙ | n = 1 − 6 |
| Glu-Glu | |
| Glu-Lys | |
| Lys-Lys | |
| ⁺NH₃-(Lys)⁺-(Gly)ₙ-(Glu)⁻-COO⁻ | n = 1 − 5 |
| ⁺NH₃-(Lys)⁺-(Gly)ₙ-(Lys)⁺-CONH₂ | n = 1 − 5 |
| CH₃—NH₂-(Glu)⁻-(Gly)ₙ-(Glu)⁻-COO⁻ | n = 1 − 5 |

In the first library, charged moieties (amino and carboxyl groups) were sequentially spaced by methylene groups. Thus, a series of dianions, dications and (non-alpha) amino acids were tested. In the second library, peptides were synthesized containing either simple short glycine polymers or lysine and glutamic acid residues spaced by glycines. In two series, peptides were either methylated at the N-terminus or amidated on the C-terminal side to create completely anionic or cationic species. Note that these modified peptides have two charges on the opposite side from the chemical modification in each case.

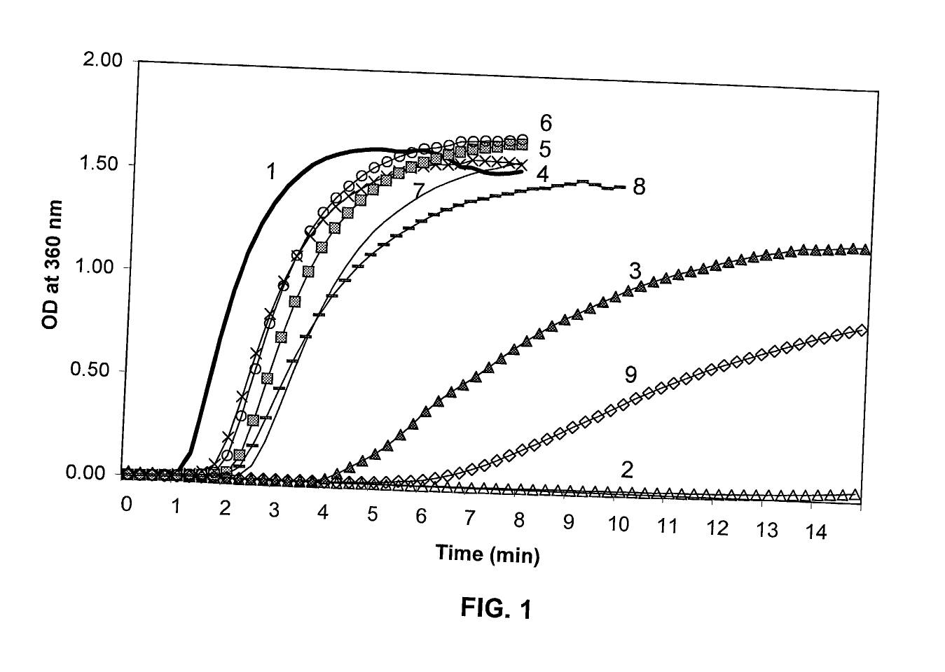

Proteins were selected that necessitated induction of aggregation by a number of different methods including reduction and oxidation as well as thermal stress. But in all cases, it was aggregation that was monitored as the stability endpoint. A typical example of the results obtained is shown in FIG. 1 which illustrates the effect of a 10,000X molar excess of dianions on the aggregation of 1 mg/mL (7.1 μM) yeast alcohol dehydrogenase at 37° C. The dianions tested include oxalate, malonate, succinate, glutarate, adipate, pimelate, suberate, and azelaate. In the graph of FIG. 1, protein is indicated by the graph line marked 1, oxalate is line number 2, malonate is line number 3, succinate is line number 4, glutarate is line number 5, adipate is line number 6, pimelate is line number 7, suberate is line number 8, and azelaate is line number 9. The oxidation-induced aggregation of alcohol dehydrogenase was found to be inhibited by a variety of compounds but most distinctly by several dicarboxylic acids. The results shown in FIG. 1 indicate that oxalate is the most potent inhibitor. However, as shown below in Table 2, the inhibition index for this agent (I=27) is actually less than that of azelaate (I=40).

TABLE 2
Effect of multiply charged compounds on the inhibition index of proteins
Protein

| | | | α | | | | |
|---|---|---|---|---|---|---|---|
| | Insulin | Lysozyme | Lactalbumin | FGF-1² | FGF-1 | HLF² | HLF |
| compound | 100× | 1,000× | 1,000× | 1,000× | 10,000× | 10,000× | 10,000× |
| oxalate | 0.9 (0.6)³ | 1.6 (1.0) | 1.3 (0.2) | 5.2 (1.5) | 605.5 (89.1) | N.T. | 666.9 |
| | | | | | | | (314.8) |
| malonate | 1.2 (0.5) | 1.5 (0.4) | 1.8 (0.1) | 2.2 (0.4) | 36.6 (29.0) | N.T. | 1714.1 |
| | | | | | | | (189.1) |
| succinate | 1.0 (0.3) | 1.0 (0.3) | 1.1 (0.3) | 2.1 (0.4) | 9.2 (1.9) | N.T. | 33.7 (11.9) |
| glutarate | 1.0 (0.2) | 1.2 (0.3) | 1.1 (0.2) | 3.0 (0.9) | 10.8 (8.4) | N.T. | 49.7 (28.9) |
| adipate | 1.2 (0.7) | 1.4 (0.8) | 1.2 (0.3) | 3.0 (0.7) | 5.0 (3.3) | N.T. | 113.0 |
| | | | | | | | (119.5) |
| pimelate | 1.4 (0.6) | 2.0 (0.7) | 1.3 (0.3) | 3.1 (1.0) | 12.1 (8.8) | N.T. | 78.9 (68.2) |
| suberate | 1.6 (0.3) | 1.4 (0.2) | 2.0 (0.7) | 2.3 (0.5) | 7.1 (5.4) | N.T. | 95.4 (34.6) |
| azelaate | 1.5 (0.4) | 1.0 (0.2) | 2.3 (0.6) | 1.9 (0.2) | 6.1 (2.6) | N.T. | 1455.0 |
| | | | | | | | (437.7) |
| guanidine HCl | 0.9 (0.4) | 0.9 (0.2) | 1.7 (0.6) | 1.2 (0.2) | 2.8 (0.9) | N.T. | 0.9 (0.1) |
| ammonium formate | 1.2 (0.5) | 1.5 (0.3) | 0.8 (0.3) | 0.9 (0.1) | 1.3 (0.3) | N.T. | 5.8 (1.1) |
| glycine | 1.0 (0.3) | 1.0 (0.2) | 1.4 (0.3) | 1.3 (0.3) | 3.0 (1.7) | N.T. | 1.5 (0.1) |
| beta-alanine | 1.1 (0.1) | 1.1 (0.3) | 2.1 (0.4) | 1.1 (0.2) | 1.5 (0.5) | N.T. | 24.2 (4.4) |
| 4-aminobuytrate | 1.2 (0.3) | 1.4 (0.4) | 2.0 (0.8) | 1.5 (0.0) | 1.4 (0.3) | N.T. | 9.5 (1.9) |
| 5-aminovalerate | 1.2 (0.1) | 1.5 (0.4) | 2.1 (0.8) | 1.1 (0.2) | 2.2 (1.4) | N.T. | 8.4 (2.6) |
| 6-aminocaproate | 1.4 (0.2) | 0.8 (0.2) | 2.3 (0.6) | 2.2 (0.3) | 2.4 (0.5) | N.T. | 7.9 (3.4) |
| 7-aminoheptanoate | 2.4 (1.0) | 1.3 (0.2) | 3.9 (2.1) | 0.9 (0.4) | 1.3 (0.1) | N.T. | 4.7 (0.7) |
| hydrazine | 0.9 (0.4) | 12.2 (6.0) | 0.5 (0.5) | 1.6 (0.6) | 7.0 (1.2) | N.T. | 16.0 (6.2) |
| ethylenediamine | 0.9 (0.3) | 70.2 (6.8) | 0.7 (0.4) | 2.2 (0.7) | 3.3 (1.3) | N.T. | 44.5 (13.9) |
| 1,3-diaminopropane | 1.3 (0.3) | 2.7 (0.4) | 0.9 (0.3) | 2.2 (0.3) | 3.5 (2.3) | N.T. | 31.5 (12.0) |
| 1,4-diaminobutane | 1.4 (0.4) | 1.6 (0.2) | 1.0 (0.2) | 2.5 (1.3) | 33.7 (6.6) | N.T. | 34.5 (13.0) |
| 1,5-diaminopentane | 1.5 (0.6) | 1.3 (0.3) | 0.9 (0.0) | 3.3 (0.7) | 46.5 (19.8) | N.T. | 28.8 (13.3) |
| 1,6-diaminohexane | 1.5 (0.3) | 1.0 (0.1) | 1.3 (0.4) | 1.6 (0.6) | 27.0 (9.8) | N.T. | 18.0 (4.9) |
| 1,7-diaminoheptane | 2.7 (1.0) | 1.0 (0.3) | 1.8 (0.7) | 2.0 (0.7) | 6.2 (2.4) | N.T. | 11.0 (2.1) |
| Glu-Glu | 1.0 (0.1) | 1.2 (0.7) | 1.0 (0.1) | 1.3 (0.2) | 7.1 (2.7) | 9.8 (0.8) | 2.1 |
| Glu-Lys | 1.0 (0.1) | 1.0 (0.4) | 1.0 (0.1) | 1.6 (0.3) | 11.0 (3.0) | 9.8 (0.3) | 44.5 |
| Lys-Lys | 1.1 (0.1) | 1.3 (0.1) | 1.0 (0.1) | 1.1 (0.1) | 3.1 (0.8) | 13.5 (1.2) | 11.2 |
| diglycine | 1.0 (0.2) | 0.9 (0.0) | 1.0 (0.0) | 1.0 (0.1) | 1.2 (0.4) | 7.4 (1.0) | 10.0 |
| triglycine | 1.4 (0.1) | 1.6 (0.3) | 0.9 (0.1) | 2.2 (1.1) | 3.1 (0.9) | 0.2 (0.1) | 5.7 (4.0) |
| tetraglycine | 1.5 (0.3) | 2.1 (0.5) | 1.3 (0.4) | 3.8 (0.8) | N.T. | 8.3 (9.5) | 0.1 |
| pentaglycine | 1.0 (0.2) | 0.2 (0.1) | 3.1 (1.2) | N.T. | N.T. | 979.0 (7.5) | 0.5 (0.0) |
| hexaglycine | 1.5 (0.7) | 0.0 (0.0) | 4.2 (1.1) | N.T. | N.T. | 1.7 (1.5) | N.T. |
| ⁺NH₃—(K)⁺—(G)₁—(K)⁺—CONH₂ | 0.9 (0.1) | 0.9 (0.2) | 0.5 (0.2) | 2.3 (0.4) | N.T. | 1.5 (0.2) | N.T. |
| ⁺NH₃—(K)⁺—(G)₂—(K)⁺—CONH₂ | 1.8 (0.1) | 0.4 (0.1) | 3.3 (0.9) | 1.8 (0.3) | N.T. | 1.3 (0.1) | N.T. |
| ⁺NH₃—(K)⁺—(G)₃—(K)⁺—CONH₂ | 0.9 (0.3) | 1.2 (0.7) | 0.8 (0.1) | 1.5 (0.4) | N.T. | 2.1 (0.1) | N.T. |
| ⁺NH₃—(K)⁺—(G)₄—(K)⁺—CONH₂ | 1.6 (0.3) | 1.4 (0.5) | 1.4 (0.3) | 2.0 (1.0) | N.T. | 1.9 (0.2) | N.T. |
| ⁺NH₃—(K)⁺—(G)₅—(K)⁺—CONH₂ | 1.7 (1.0) | 0.8 (0.4) | 2.6 (1.1) | 3.3 (0.8) | N.T. | 1.5 (0.3) | N.T. |
| CH₃—NH₂—(E)⁻—(G)₁—(E)⁻—COO⁻ | 0.5 (0.2) | 0.9 (0.1) | 0.2 (0.1) | 1.9 (0.1) | N.T. | 1.6 (0.1) | N.T. |
| CH₃—NH₂—(E)⁻—(G)₂—(E)⁻—COO⁻ | 1.0 (0.2) | 0.8 (0.4) | 0.4 (0.2) | 2.5 (0.6) | N.T. | 1.7 (0.2) | N.T. |
| CH₃—NH₂—(E)⁻—(G)₃—(E)⁻—COO⁻ | 1.3 (0.4) | 0.9 (0.2) | 0.5 (0.3) | 2.0 (0.3) | N.T. | 1.5 (0.3) | N.T. |
| CH₃—NH₂—(E)⁻—(G)₄—(E)⁻—COO⁻ | 1.4 (0.6) | 0.9 (0.3) | 0.7 (0.4) | 1.6 (0.2) | N.T. | 1.7 (0.3) | N.T. |
| CH₃—NH₂—(E)⁻—(G)₅—(E)⁻—COO⁻ | 1.3 (0.1) | 0.1 (0.1) | N.T.¹ | 12.3 (0.4) | N.T. | 1.6 (0.1) | N.T. |
| KGE | 0.6 (0.3) | 2.8 (0.8) | 0.3 (0.2) | 1.7 (0.4) | N.T. | 0.9 (0.1) | N.T. |
| KGGE | 1.0 (0.3) | 2.6 (0.4) | 0.5 (0.2) | 1.2 (0.2) | N.T. | 0.7 (0.1) | N.T. |
| KGGGE | 0.8 (0.1) | 3.4 (0.1) | 0.6 (0.1) | 1.9 (0.2) | N.T. | 0.8 (0.1) | N.T. |
| KGGGGE | 1.0 (0.1) | 1.9 (0.5) | 0.5 (0.1) | 1.9 (0.4) | N.T. | 0.8 (0.1) | N.T. |
| KGGGGGE | 1.3 (0.3) | 1.5 (0.4) | 1.8 (0.2) | 1.4 (0.3) | N.T. | 0.8 (0.1) | N.T. |
| | CS² | CS | CS | ADH² | ADH | pST² | pST |
| compound | 1,000X | 10,000X | 50,000X | 1,000X | 10,000X | 100X | 2,000X |
| oxalate | N.T. | 1.9 (0.5) | 6.2 (3.8) | N.T. | 36.7 (3.1) | 0.8 (0.1) | 1.5 (0.3) |
| malonate | N.T. | 2.4 (0.5) | 9 | N.T. | 18.9 (6.6) | 0.9 (0.1) | 4.9 (0.5) |
| succinate | N.T. | 2.1 (0.5) | 9.6 | N.T. | 2.7 (0.7) | 0.9 (0.1) | 2.1 (0.3) |
| glutarate | N.T. | 1.4 (0.5) | 6.5 (0.3) | N.T. | 1.8 (0.4) | 0.9 (0.1) | 3.1 (0.7) |
| adipate | N.T. | 1.2 (0.5) | 19.6 (17.1) | N.T. | 4.9 (0.9) | 0.9 (0.1) | 3.9 (0.7) |
| pimelate | N.T. | 1.4 (0.4) | 6.7 (0.1) | N.T. | 9.0 (1.3) | 0.8 (0.1) | 1.5 (0.4) |
| suberate | N.T. | 1.1 (0.2) | 14.1 (2.1) | N.T. | 15.5 (1.4) | 0.7 (0.0) | 1.7 (0.3) |
| azelaate | N.T. | 0.7 (0.6) | 4.4 (1.8) | N.T. | 40.1 (5.2) | 0.6 (0.0) | 1.3 (0.3) |
| guanidine HCl | N.T. | 0.6 (0.4) | 0.4 (0.1) | N.T. | 0.2 (0.1) | N.T. | N.T. |
| ammonium formate | N.T. | 0.6 (0.0) | 1.2 (0.3) | N.T. | 1.2 (0.1) | N.T. | N.T. |
| glycine | N.T. | 0.8 (0.2) | N.T. | N.T. | 54.3 (4.5) | 0.4 (0.1) | 1.2 (0.2) |
| beta-alanine | N.T. | 0.6 (0.1) | 1.9 (0.8) | N.T. | 2.2 (0.3) | 0.4 (0.1) | 0.8 (0.1) |
| 4-aminobuytrate | N.T. | 0.6 (0.1) | 1.2 (0.4) | N.T. | 2.0 (0.1) | 0.3 (0.0) | 1.1 (0.2) |
| 5-aminovalerate | N.T. | 0.7 (0.1) | 1.9 (0.7) | N.T. | 2.7 (0.9) | 0.4 (0.0) | 0.7 (0.1) |
| 6-aminocaproate | N.T. | 0.5 (0.1) | 1.5 (0.8) | N.T. | 3.2 (0.3) | 0.5 (0.1) | 1.2 (0.2) |
| 7-aminoheptanoate | N.T. | 0.5 (0.1) | 1.7 (0.4) | N.T. | 4.9 (0.5) | 0.4 (0.0) | 1.6 (0.2) |
| hydrazine | N.T. | 0.9 (0.2) | 6.8 | N.T. | 0.3 (0.1) | N.T. | N.T. |
| ethylenediamine | N.T. | 1.8 (0.5) | 18.5 | N.T. | 202.6 (69.7) | 0.7 (0.0) | 1.3 (0.1) |
| 1,3-diaminopropane | N.T. | 0.7 (0.3) | 7.7 | N.T. | 0.7 (0.1) | 0.6 (0.0) | 1.7 (0.2) |
| 1,4-diaminobutane | N.T. | 0.9 (0.3) | 4.0 (2.3) | N.T. | 0.8 (0.1) | 0.6 (0.1) | 1.4 (0.4) |
| 1,5-diaminopentane | N.T. | 0.8 (0.2) | 8.3 (4.5) | N.T. | 0.9 (0.0) | 0.8 (0.3) | 1.7 (0.4) |
| 1,6-diaminohexane | N.T. | 0.6 (0.2) | 4.8 (1.5) | N.T. | 0.8 (0.0) | 0.6 (0.1) | 1.9 (0.2) |
| 1,7-diaminoheptane | N.T. | 0.6 (0.2) | 1.7 (0.7) | N.T. | 0.6 (0.1) | 0.7 (0.0) | 1.6( 0.7) |
| Glu-Glu | 0.8 (0.1) | 1.5 (0.1) | 1430.3 | 1.4 (0.2) | 5.3 (1.2) | 0.6 (0.1) | N.T. |
| Glu-Lys | 0.8 (0.2) | 1.6 (0.2) | 21.5 | 1.7 (0.1) | 18.4 | N.T. | N.T. |
| Lys-Lys | 0.9 (0.1) | 1.6 (0.3) | 86.8 | 1.4 (0.2) | 2.7 | 0.9 (0.1) | N.T. |
| diglycine | 0.8 (0.1) | 0.9 (0.3) | 1.5 (0.2) | 4.0 (0.9) | 0.7 (0.1) | 1.9 (0.3) | |
| triglycine | 1.0 (0.2) | 0.7 (0.2) | 1.0 | 5.0 (1.4) | 187.6 (46.1) | 0.7 (0.0) | 2.5 (0.3) |
| tetraglycine | 1.5 (0.1) | N.T. | N.T. | 8.4 (2.7) | 5313.1 | 0.8 (0.1) | 3.4 (0.6) |
| pentaglycine | 0.6 (0.2) | N.T. | N.T. | 5.8 (3.1) | Infinite⁴ | 0.7 (0.2) | N.T. |
| hexaglycine | 0.5 (0.1) | N.T. | N.T. | 1.7 (0.5) | 20.3 | 0.7 (0.1) | N.T. |
| ⁺NH₃—(K)⁺—(G)₁—(K)⁺—CONH₂ | 1.0 (0.3) | N.T. | N.T. | 1.2 (0.1) | N.T. | N.T. | N.T. |
| ⁺NH₃—(K)⁺—(G)₂—(K)⁺—CONH₂ | 0.7 (0.2) | N.T. | N.T. | 0.8 (0.1) | N.T. | 1.2 (0.2) | N.T. |
| ⁺NH₃—(K)⁺—(G)₃—(K)⁺—CONH₂ | 0.6 (0.1) | N.T. | N.T. | 1.2 (0.1) | N.T. | N.T. | N.T. |
| ⁺NH₃—(K)⁺—(G)₄—(K)⁺—CONH₂ | 0.8 (0.2) | N.T. | N.T. | 1.5 (0.3) | N.T. | 0.9 (0.0) | N.T. |
| ⁺NH₃—(K)⁺—(G)₅—(K)⁺—CONH₂ | 0.8 (0.1) | N.T. | N.T. | 1.5 (0.2) | N.T. | N.T. | N.T. |
| CH₃—NH₂—(E)⁻—(G)₁—(E)⁻—COO⁻ | 0.8 (0.1) | N.T. | N.T. | 1.6 (0.1) | N.T. | 1.0 (0.1) | N.T. |
| CH₃—NH₂—(E)⁻—(G)₂—(E)⁻—COO⁻ | 0.9 (0.2) | N.T. | N.T. | 1.1 (0.1) | N.T. | N.T. | N.T. |
| CH₃—NH₂—(E)⁻—(G)₃—(E)⁻—COO⁻ | 1.0 (0.2) | N.T. | N.T. | 1.3 (0.1) | N.T. | 1.2 (0.2) | N.T. |
| CH₃—NH₂—(E)⁻—(G)₄—(E)⁻—COO⁻ | 0.7 (0.1) | N.T. | N.T. | 1.4 (0.1) | N.T. | N.T. | N.T. |
| CH₃—NH₂—(E)⁻—(G)₅—(E)⁻—COO⁻ | 0.6 (0.1) | N.T. | N.T. | 1.2 (0.1) | N.T. | N.T. | N.T. |
| KGE | 1.2 (0.2) | N.T. | N.T. | 1.3 (0.1) | N.T. | 0.7 (0.0) | N.T. |
| KGGE | 1.4 (0.4) | N.T. | N.T. | 0.8 (0.1) | N.T. | 0.6 (0.1) | N.T. |
| KGGGE | 1.5 (0.1) | N.T. | N.T. | 1.2 (0.1) | N.T. | 0.6 (0.1) | N.T. |
| KGGGGE | 1.8 (0.2) | N.T. | N.T. | 1.4 (0.1) | N.T. | 0.6 (0.1) | N.T. |
| KGGGGGE | 2.0 (0.2) | N.T. | N.T. | 1.4 (0.2) | N.T. | 0.5 (0.1) | N.T. |

¹N.T. = not testing due to lack of solubility, compound availability, pH problems or minimal effects at lower concentrations.
²FGF-1 = acidic fibroblast growth factor, HLF = human lactoferrin, CS = citrate synthase, ADH = alcohol dehydrogenase, pST = porcine somatotopin
³The number in parenthesis represents the standard deviation of the inhibition index.
⁴infinite—complete inhibition of aggregation over a one-hour period of examination.

This difference reflects the rapid rate and greater extent of aggregation seen at the longer times not shown in the figure. Note that these effects are not due to simple chelation of copper which was employed in a chelated form or to ionic strength because NaCl at this ionic strength has negligible effect on aggregation. Very close pH control was also used in all experiments to eliminate direct effects of pH on the association processes of experiments. Potent inhibition of ADH was also seen with glycine and to a lesser extent with several non alpha amino acids. The longer glycine peptides were also strong inhibitors.

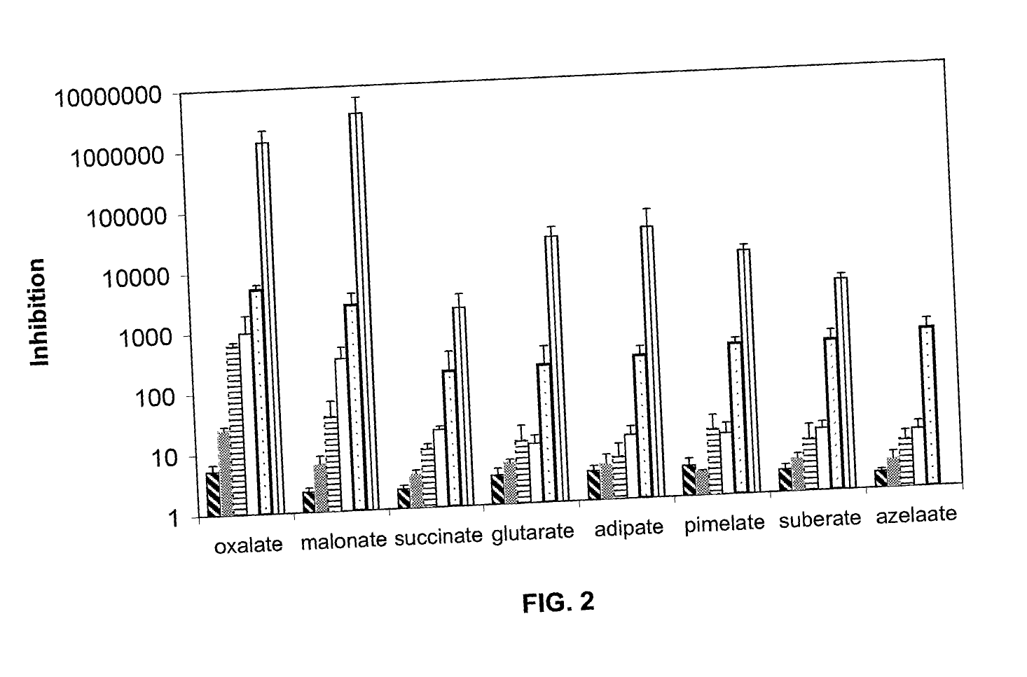

Results for 8 proteins are summarized in Table 2. In some cases, little inhibition was seen as in the case of insulin although even in this case, weak inhibitions were identified (i.e., 1,6-diaminoheptane and 7-aminoheptanoate). Potent inhibitors were identified for lysozyme (small diamines and several KGₙE peptides), α-lactalbumin (G5, G6, C7, KG₅K), FGF-1 (dicarboxylic acids, diamines, several peptides), lactoferrin (some small peptides), citrate synthase (diacids, diamines, EE, EK, GG, GGG) and porcine somatotropin (several diacids, diamines, larger Gₙ). ADH was inhibited by the diacids. Careful inspection of Table 2 reveals no clear pattern as expected if the compounds are weakly being bound to one or more sites on the protein surface. As an example, FIG. 2 illustrates the effect of inhibitor concentration for dianions on FGF-1 aggregation. To prepare this figure, a 50 μg/mL solution of FGF-1 in PBS was monitored at 50° C. In this figure, starting from left to right for the series of bars corresponding to each protein, ligand concentrations are 1,000X, 5,000X, 10,000X, 12,500X, 25,000X, and 50,000X. As diacid concentration increases, the inhibition increases as expected. Conversely, in only a few cases did increased ligand concentrations destabilize any of the proteins under the conditions tested. Among the ligands tested, four stood out as effective inhibitors: oxalate (FGF-1, ADH), glycine (ADH), ethylenediamine (lysozyme, ADH) and beta-alanine (HLF). Within a series of dipolar compounds of the same type, a noticeable effect of charge density can be seen with FGF-1. In contrast, lysozyme is strongly inhibited by the smaller hydrazine and ethylenediamine dications. In general, inhibitory concentrations of polyions were typically in the range of micro to millimolar, indicating a moderate affinity for the binding process.

Discussion.

The present invention provides a method for rapidly identifying stabilizers of proteins in aqueous solution. The entire library described can be screened in less than an hour in a microtiter plate, thereby making it extremely simple and rapid. Simple compounds and peptides with charged termini spaced to varying degrees were chosen due to their potential for occasionally having the appropriate geometry to cross-link unpaired charges on protein surfaces. In preliminary studies, charged polyethylene glycols of the form A-(CH₂—CH₂—O)ₙ-A wherein each A is independently selected from the group consisting of NH₃⁺ and COO⁻, were also found to occasionally be good inhibitors although more often they accelerated the aggregation process. Such protein ligand interactions were predicted to stabilize proteins to varying degrees by preferential binding to the native protein structure compared to structurally altered states. Such interactions could be thought of as “locking” or “stapling” proteins into their more bioactive forms. Destabilization would be expected if polyions bound better to non-native forms and this is in fact observed in some cases (i.e., I<1 in Table 2). It is possible that compounds identified in this manner might themselves be useful as stabilizing agents for pharmaceutical formulations of proteins since inhibitory effects are seen at fairly moderate ligand concentrations. Preliminary studies indicate inhibitors do not affect biological activity in most cases and are not toxic to cells in culture at the concentrations employed. The former is expected because dilution upon introduction to biological targets would be expected to cause dissociation of ligands from protein surfaces. We think equally likely, however, that the compounds initially identified could serve as starting points for new libraries that can be used to obtain higher affinity ligands. Thus a skeleton defined by charged groups at the defined spacing could be chemically varied in their spacing regions to add additional interactions with a protein's surface.

The most striking finding here is that the time-dependent aggregation of all of the proteins examined is inhibited by one or more of the charged compounds to varying degrees. This inhibition is often quite selective and can involve compounds apparently unrelated in structure. This is, again, consistent with binding of the compounds to specific sites on each protein. Preliminary studies indicate that such interactions can, in fact, be detected in many cases by a variety of biophysical methods. FGF-1 presents an interesting test of this approach because the protein is known to have a rather promiscuous polyanion binding site, the occupation of which dramatically stabilizes the protein (Volkin, D. B. & Middaugh, C. R. (1996) Pharm. Biotechnol. 9, 181-217, the content and teachings of which are hereby incorporated by reference herein). The inhibition of aggregation of FGF-1 by all members of the diacid series presumably reflects binding to this site with the specificity exhibited (FIG. 2) which is a function of the detailed geometry of this polycationic region. Not predictable, however, is the potent inhibition seen with several diamines and peptides. This again validates the ability of this screening methodology to identify selective modifiers of protein stability.

This approach is certainly not limited to the simple turbiditymethod employed. Light scattering based measurements have the advantage that they presumably directly detect the presence of molten-globule-like states that are thought to generally be responsible for a wide variety of instabilities (Fink, A. L. (1998) Fold Des. 3, R9-23, Fink, A. L. (1995) Ann. Rev. Biophys. Biomol. Struct. 24, 495-522, the content and teachings of which are hereby incorporated by reference herein). Use of other methods to detect loss of stability such as changes in intrinsic protein fluorescence or the binding of polarity sensitive dyes such as ANS should also be useful as well since they are easily adaptable to a microtiter plate format. In many but not all cases, accelerated stability studies of this type are reasonably predictive of protein stability behavior under more moderate conditions (Yoshioka, S. & Stella, V. J. (2000) Stability of Drugs and Dosage Forms (Kuwer Academic/Plenum Pub., New York, the content and teachings of which are hereby incorporated by reference herein).

When dye binders are used, changes in fluorescence can be used to determine the effect of compounds in accordance with the present invention on protein stability. This is because proteins have a well defined three-dimensional tertiary structure which, when stressed, swells into an intermediate state known as the molten globule state. If the protein is stressed further, it unfolds. However, the molten globule state is believed to be the state in which proteins aggregate. Dye binders bind to this molten globule state, thereby changing the fluorescence of the dye. Accordingly, in order to determine the effect of a compound on the stability of a protein, a protein with the added compound is compared to a protein without the added compound by adding a dye such as ANS or bis-ANS to each protein. Increases in fluorescence indicate that the protein has progressed to the molten globule state and is therefore, not stable. If the protein has become entirely unfolded or has remained in its original tertiary structure, the dye binder will not bind as well to the protein and the fluorescence will not change over time. For purposes of the present invention, any dye binder such as 1-anilinonaphthalene-8-sulfonic acid (1, 8-ANS), 2,6-ANS, bis-ANS (4,4′-dianilino-1,1′-binaphthyl-5,5′-disulfonic acid, dipotassium salt), 4-(dicyanovinyl)julolidine (DCVJ), dansyl lysine (N-e-(5-dimethylaminonaphthalene-1-sulfonyl)-L-lysine), laurdan (6-dodecanoyl-2-dimethylaminonaphthalene), patman (6-hexadecanoyl-2-(((2-(trimethylammonium)ethyl)methyl)amino)naphthalene chloride), Nile red, N-phenyl-1-naphthylamine, prodan (5-propionyl-2-dimethylaminonaphthalene) and 2-(p-toluidinyl) naphthalene-6-sulfonic acid, sodium salt (2, 6-TNS), and combinations thereof can be used. Preferably, such dye binders will be negatively charged and a preferred group includes the ANS family of dye binders because of their common usage in the art.

Compounds identified in this manner could potentially have other uses. They could be employed to enhance protein folding or used to minimize aggregation problems during protein isolation procedures. This approach could also be extended to the stabilization of more complex molecular entities such as vaccines and gene delivery vehicles (Gibson, T. D. (1996) Dev. Biol. Stand. 87, 207-217; Dorval, B. L., Chow, M. & Klibanov, A. M. (1990) Biotechnol. Bioeng. 35, 1051-1054, the content and teachings of which are hereby incorporated by reference herein). Finally, it should also be possible to identify inhibitors of pathological protein aggregation as manifested in many disease states such as Alzheimers, Parkinson's, and sickle cell disease, cataract formation, other amyloid related disorders, spongiform encephalopathies such as mad cow disease, and polyglutamine-based pathologies such as Huntington's chorea.

The preferred forms of the invention described above are to be used as illustration only, and should not be utilized in a limiting sense in interpreting the scope of the present invention. Obvious modifications to the exemplary embodiments and examples, as set forth herein, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

What is claimed is:

1. A method of altering the stability of a protein comprising the step of:
contacting said protein with a compound, said compound comprising a molecule having more than one charge.

2. The method of claim 1, said molecule being selected from the group consisting of di-ions, tri-ions, and tetra-ions.

3. The method of claim 2, said di-ions, tri-ions, and tetra-ions being selected from the group consisting of oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, hexaglycine, ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃—NH₂-(Glu)⁻-(Gly)ₙ-(Glu)⁻-COO⁻, and combinations thereof, wherein n=1 to 5.

4. The method of claim 1, said contacting step resulting in an increase in protein stability.

5. The method of claim 1, said contacting step resulting in a decrease in protein stability.

6. The method of claim 1, said protein stability being measured by the effect of said compound on the aggregation of said protein in solution.

7. The method of claim 6 said aggregation being measured by determining the change of turbidity of said solution after said contacting step.

8. The method of claim 1, said compound preferentially interacting with the native state of said protein.

9. The method of claim 1, said compound binding to unpaired charged sites on the surface of said protein.

10. The method of claim 1, said protein being used in a pharmaceutical preparation.

11. The method of claim 1, said compound having a molecular weight of less than about 2000.

12. A method of altering protein aggregation comprising the steps of:
providing a protein in solution;
contacting said protein with a compound, said compound comprising a molecule which contains more than one charge.

13. The method of claim 12, said molecule being selected from the group consisting of di-ions, tri-ions, and tetra-ions.

14. The method of claim 13, said di-ions, tri-ions, and tetra-ions being selected from the group consisting of oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, hexaglycine, ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃-NH₂-(Glu)⁻-(Gly)ₙ-(Glu)⁻-COO⁻, and combinations thereof, wherein n=1 to 5.

15. The method of claim 12, said contacting step resulting in an increase in protein stability.

16. The method of claim 12, said contacting step resulting in a decrease in protein stability.

17. The method of claim 12, said protein stability being measured by the effect of said compound on the aggregation of said protein in solution.

18. The method of claim 17 said aggregation being measured by determining the change of turbidity of said solution after said contacting step.

19. The method of claim 12, said compound preferentially interacting with the native state of said protein.

20. The method of claim 12, said compound binding to unpaired charged sites on the surface of said protein.

21. The method of claim 12, said protein being used in a pharmaceutical preparation.

22. The method of claim 12, said compound having a molecular weight of less than about 2000.

23. A method of screening compounds which alter the stability of proteins, said method comprising the steps of:
providing a protein in solution;
adding a compound to said solution, said compound comprising a molecule which contains more than one charge;
adding a stability altering agent to said solution; and
determining the effect of said agent on the stability of said protein.

24. The method of claim 23, said determining step including the step of testing the stability of said protein in solution before adding said compound to said solution.

25. The method of claim 23, said determining step including the step of testing the stability of said protein in solution after the addition of said stability altering agent.

26. The method of claim 23, said determining step including the steps of
testing the stability of said protein after the addition of said compound to provide a first stability;
testing the stability of said protein before the addition of said compound to provide a second stability; and
comparing said first stability to said second stability.

27. The method of claim 26, further comprising the step of adding the stability altering agent prior to stability testing.

28. The method of claim 23, said molecule being selected from the group consisting of di-ions, tri-ions, and tetra-ions.

29. The method of claim 28, said di-ions and tri-ions being selected from the group consisting of oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, hexaglycine, ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃—NH₂-(Glu)⁻-(Gly)ₙ-(Glu)⁻COO⁻, and combinations thereof, wherein n=1 to 5.

30. The method of claim 23, wherein the addition of said compound results in an increase in protein stability.

31. The method of claim 23, wherein the addition of said compound results in a decrease in protein stability.

32. The method of claim 23, said protein stability being measured by the effect of said compound on the aggregation of said protein in solution.

33. The method of claim 32, said aggregation being measured by determining the change of turbidity of said solution after said compound has been added.

34. The method of claim 23, said compound preferentially interacting with the native state of said protein.

35. The method of claim 23, said compound binding to unpaired charged sites on the surface of said protein.

36. The method of claim 23, said protein being used in a pharmaceutical preparation.

37. The method of claim 23, said compound having a molecular weight of less than about 2000.

38. A protein having an altered stability, said protein having a ligand bound thereto, said ligand comprising a molecule having more than one charge, wherein said protein without said ligand bound thereto exhibits a first stability and said protein with said ligand bound thereto exhibits a second stability.

39. The protein of claim 38, said molecule being selected from the group consisting of di-ions, tri-ions, and tetra-ions.

40. The protein of claim 39, said di-ions, tri-ions, and tetra-ions being selected from the group consisting of oxalic acid, sodium malonate, succinic acid, glutaric acid, adipic acid, pimelic acid, suberic acid, azelaic acid, guanidine hydrochloride, ammonium formate, beta-alanine, 4-aminobutyric acid, 5-aminovaleric acid, 6-aminocaproic acid, 7-aminoheptanoic acid, hydrazine, ethylenediamine, 1,3-diaminopropane, 1,4-diaminobutane, 1,5-diaminopentane, 1,6-diaminohexane, 1,7-diaminoheptane, Glu-Glu, Glu-Lys, Lys-Lys, diglycine, triglycine, tetraglycine, pentaglycine, hexaglycine, ⁺NH₃-(Lys)⁺-(Gly)ₙ-(Glu)⁻-COO⁻, ⁺NH₃-(Lys)⁺-(Gly)ₙ-(Lys)⁺-CONH₂, CH₃—NH₂-(Glu)⁻-(Gly)ₙ-(Glu)—COO⁻, and combinations thereof, wherein n=1 to 5.

41. The protein of claim 38, said protein second stability being higher than said protein first stability.

42. The protein of claim 38, said protein second stability being lower than said protein first stability.

43. The protein of claim 38, said first and second protein stabilities being measured by the effect of said compound on the aggregation of said protein in a solution.

44. The protein of claim 43, said aggregation being measured by determining the change of turbidity of said solution.

45. The protein of claim 38, said ligand preferentially interacting with the native state of said protein.

46. The protein of claim 38, said ligand binding to unpaired charged sites on the surface of said protein.

47. The protein of claim 38, said protein being used in a pharmaceutical preparation.

48. The protein of claim 38, said ligand having a molecular weight of less than about 2000.

49. A ligand which binds to a protein, said ligand having the formula q-(Gly)ₙ-q, wherein q represents a charged amino acid and n is from 1 to 6.

50. The ligand of claim 49, wherein each q is respectively and independently selected from the group consisting of lysine, arginine, histidine, aspartic acid, and glutamic acid.

51. The ligand of claim 49, said ligand operable for binding to a protein.

52. The ligand of claim 51, said protein having unpaired charge sites on its surface.

53. The ligand of claim 50, said ligand having a molecular weight of less than about 2000.

54. The ligand of claim 50, said ligand being modified by being methylated at the N-terminus.

55. The ligand of claim 50, said ligand being modified by being amidated on the C-terminal side.

56. A method of determining the effects of a compound on a protein, said compound comprising a molecule having more than one charge, said method comprising the steps of:
adding said protein to a first solution;
adding said protein and said compound to a second solution;
adding a dye binder to said first and said second solutions;
measuring the fluorescence of said first and said second proteins; and
comparing said fluorescence of said first protein with said fluorescence of said second protein.

57. The method of claim 56, said dye binder being selected from the group consisting of ANS, 4-(dicyanovinyl) julolidine, dansyl lysine, laurdan, patman, Nile red, N-phenyl-1-naphthylamine, prodan and 2-(p-toluidinyl) naphthalene-6-sulfonic acid, sodium salt, and combinations thereof.

58. The method of claim 57, said dye binder being ANS.

59. The method of claim 58, said ANS being selected from the group consisting of 1,8-ANS, 2,6-ANS, bis-ANS, and combinations thereof.

60. The method of claim 56, said method further including the step of stressing said protein.

61. The method of claim 60, said stressing including the step of altering the environment of said solution.

62. The method of claim 61, said environment being altered by a condition or agent selected from the group consisting of elevated temperatures, altering the pH of the solution, adding a reducing agent, adding an oxidizing agent, adding a chaotropic agent, and combinations thereof.

63. A method of determining the effects of a compound on a protein, said compound comprising a molecule having more than one charge, said method comprising the steps of:
adding said protein to a first solution;
adding said protein and said compound to a second solution;
measuring the intrinsic fluorescence of said first and said second proteins; and
comparing said intrinsic fluorescence of said first protein with said intrinsic fluorescence of said second protein.
